(19) United States
(12) Patent Application Publication
(10) Pub. No.: US 2023/0140585 A1
(43) Pub. Date: May 4, 2023

(54) MULTIPLE SENSOR-FUSING BASED INTERACTIVE TRAINING SYSTEM AND MULTIPLE SENSOR-FUSING BASED INTERACTIVE TRAINING METHOD

(71) Applicant: Industrial Technology Research Institute, Hsinchu (TW)

(72) Inventors: Hung-Hsien Ko, Hsinchu County (TW); Heng-Yin Chen, Hsinchu County (TW); Chen-Tsai Yang, Taoyuan City (TW)

(73) Assignee: Industrial Technology Research Institute, Hsinchu (TW)

(21) Appl. No.: 17/975,628

(22) Filed: Oct. 28, 2022

Related U.S. Application Data
(60) Provisional application No. 63/273,160, filed on Oct. 29, 2021.

(30) Foreign Application Priority Data
Sep. 13, 2022 (TW) 111134592

(51) Int. Cl. A61B 5/11; A61B 5/00; G06K 9/62

(52) U.S. Cl. CPC A61B 5/1118; A61B 5/7275; A61B 5/1121; G06K 9/6288

(57) ABSTRACT

A multiple sensor-fusing based interactive training system, including a posture sensor, a sensing module, a computing module, and a display module, is provided. The posture sensor is configured to sense posture data and myoelectric data related to a training action. The sensing module is configured to output limb torque data according to the posture data, and output muscle group activation time data according to the myoelectric data. The computing module is configured to respectively convert the limb torque data and the muscle group activation time data into a moment-skeleton coordinate system and a muscle strength eigenvalue-skeleton coordinate system according to a skeleton coordinate system, perform fusion calculation, calculate evaluation data based on a result of the fusion calculation, and judge that the training action corresponds to a known exercise action according to the evaluation data. The display module is configured to display the evaluation data and the known exercise action.

### CROSS-REFERENCE TO RELATED APPLICATION

This application claims the priority benefit of U.S. Provisional Application No. 63/273,160, filed on Oct. 29, 2021 and Taiwan Application No. 111134592, filed on Sep. 13, 2022. The entirety of each of the above-mentioned patent applications is hereby incorporated by reference herein and made a part of this specification.

### TECHNICAL FIELD

The disclosure relates to a training system, and more particularly to a multiple sensor-fusing based interactive training system and a multiple sensor-fusing based interactive training method.

### BACKGROUND

At present, the number of people who exercise regularly is increasing, and gyms can be found almost everywhere. There are various fitness equipment in the gym. After reaching the gym, many users start using the fitness equipment only according to simple instructions on the fitness equipment, without going through guidance by the coach. There are endless cases of exercise injuries caused by improper use of the fitness equipment.

Alternatively, even after going through the guidance by the coach, when performing self-training with no coach nearby, exercise injuries may also be caused by inaccurate posture, the use of muscle groups that do not match the training action, or the inaccurate sequence of straining of muscle groups for the training action.

In addition, when an athlete is training, the coach or bystanders can only preliminarily judge whether there is a risk of exercise injuries from the action posture of the athlete. However, there is no way to quantify the exercise effectiveness as an indicator for the coach and the athlete to discuss improvement manners.

### SUMMARY

The multiple sensor-fusing based interactive training system provided by the disclosure includes a posture sensor, a sensing module, a computing module, and a display module. The posture sensor includes an inertia sensor and a myoelectric sensor. The inertia sensor is configured to sense multiple posture data related to a training action of a user, and the myoelectric sensor is configured to sense multiple myoelectric data related to the training action of the user. The sensing module is configured to output limb torque data according to the posture data, and output muscle group activation time data according to the myoelectric data. The computing module is configured to respectively convert the limb torque data and the muscle group activation time data into moment-skeleton coordinates and muscle strength eigenvalue-skeleton coordinates according to skeleton coordinates, perform fusion calculation on the moment-skeleton coordinates and the muscle strength eigenvalue-skeleton coordinates, calculate evaluation data for the training action according to a result of the fusion calculation, and judge that the training action corresponds to one of multiple known exercise actions according to the evaluation data. The display module is configured to display the evaluation data and the known exercise actions.

The multiple sensor-fusing based interactive training method provided by the disclosure includes the following steps. Multiple posture data related to a training action of a user are sensed through an inertia sensor of a posture sensor, and multiple myoelectric data related to the training action of the user are sensed through a myoelectric sensor of the posture sensor. Multiple limb torque data are output according to the posture data, and multiple muscle group activation time data are output according to the myoelectric data. The limb torque data are converted into a moment-skeleton coordinate system according to a skeleton coordinate system. The muscle group activation time data are converted into a muscle strength eigenvalue-skeleton coordinate system according to the skeleton coordinate system. Fusion calculation is performed on the moment-skeleton coordinate system and the muscle strength eigenvalue-skeleton coordinate system. Evaluation data for the training action is calculated according to a result of the fusion calculation. The training action corresponds to one of multiple known exercise actions is judged according to the evaluation data. The evaluation data and the known exercise action are displayed.

### BRIEF DESCRIPTION OF THE DRAWINGS

FIG. 1 is a structural diagram of a multiple sensor-fusing based interactive training system according to an embodiment of the disclosure.

FIG. 2 is a schematic diagram of performing fusion calculation by a computing module in a multiple sensor-fusing based interactive training system according to an embodiment of the disclosure.

FIG. 3 is a block diagram of a multiple sensor-fusing based interactive training system according to an embodiment of the disclosure.

FIG. 4 is a block diagram of a multiple sensor-fusing based interactive training system according to another embodiment of the disclosure.

FIG. 5 is a block diagram of updating training data and error data by a multiple sensor-fusing based interactive training system according to an embodiment of the disclosure.

FIG. 6 is a schematic diagram of judging an offset using an inertia sensor in a multiple sensor-fusing based interactive training system according to an embodiment of the disclosure.

FIG. 7 is a block diagram of judging an offset using an inertia sensor in a multiple sensor-fusing based interactive training system according to an embodiment of the disclosure.

FIG. 8A is a schematic diagram of calculating left-right balance using a posture sensor and a mechanical sensor in a multiple sensor-fusing based interactive training system according to an embodiment of the disclosure.

FIG. 8B is another schematic diagram of calculating left-right balance using a posture sensor and a mechanical sensor in a multiple sensor-fusing based interactive training system according to an embodiment of the disclosure.

FIG. 9 is a block diagram of calculating left-right balance using a posture sensor and a mechanical sensor in a multiple sensor-fusing based interactive training system according to an embodiment of the disclosure.

FIG. 10 is a schematic diagram of performing action recognition using a body inertia sensor and a myoelectric sensor in a multiple sensor-fusing based interactive training system according to an embodiment of the disclosure.

FIG. 11 is a flowchart of a multiple sensor-fusing based interactive training method according to an embodiment of the disclosure.

FIG. 12 is a flowchart of calculating evaluation data in a multiple sensor-fusing based interactive training method according to an embodiment of the disclosure.

### DETAILED DESCRIPTION OF DISCLOSED EMBODIMENTS

Some embodiments of the disclosure will be described in detail below with reference to the drawings. In the following description, when the same reference numeral appears in different drawings, the reference numeral is regarded as referring to the same or similar elements. The embodiments are only a part of the disclosure and do not disclose all possible implementations of the disclosure.

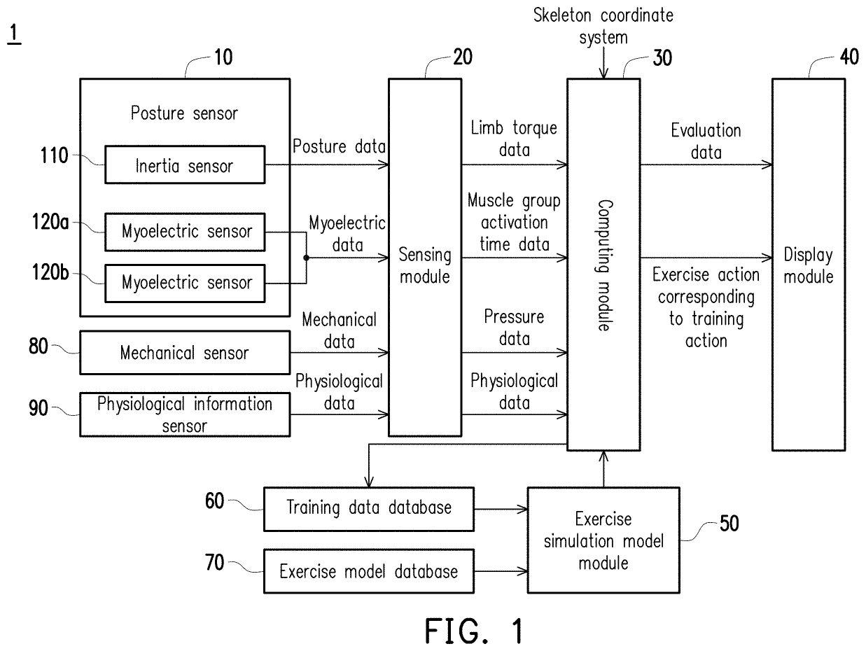

FIG. 1 is a structural diagram of a multiple sensor-fusing based interactive training system 1 according to an embodiment of the disclosure. Please refer to FIG. 1. The multiple sensor-fusing based interactive training system 1 includes a posture sensor 10, a sensing module 20, a computing module 30, and a display module 40. The multiple sensor-fusing based interactive training system 1 senses data related to a training action of a user through the posture sensor 10, and after processing the sensed data, which of the built-in exercise actions in the multiple sensor-fusing based interactive training system 1 the training action being executed by the user belongs to is judged, and data for reference by the user when performing the training action is displayed in real time. The feedback of the multiple sensor-fusing based interactive training system 1 may help the user to judge whether the posture during training is accurate, whether the main muscle groups used match the training action, whether the sequence of straining of muscle groups for the training action is accurate, etc.

The posture sensor 10 includes an inertia sensor 110, a myoelectric sensor 120a, and a myoelectric sensor 120b. The inertia sensor 110 is configured to sense multiple posture data related to the training action of the user. The inertia sensor 110 may be disposed on the body or the limbs of the user depending on the training action of the user. For example, when the user is running, the inertia sensor 110 may be disposed on positions such as the waist, the outer sides of the legs, and the shoes of the user. The posture data related to the running action include stride frequency, stride length, vertical amplitude, body inclination angle, feet contact time, feet movement trajectory, etc. The posture data are all related to the economy of running and can effectively monitor the posture of the user when running. In practice, the inertia sensor 110 is, for example, a dynamic sensor such as a gravity sensor (G-sensor), an angular velocity sensor, a gyro sensor, and a stride frequency sensor, but not limited thereto.

The myoelectric sensor 120a and the myoelectric sensor 120b are configured to sense multiple myoelectric data related to the training action of the user. The myoelectric sensor 120a and the myoelectric sensor 120b may be attached or worn above the core muscle groups or related muscle groups of the user, such as the left and right thighs, the left and right calves, the left and right arms, the back muscles on both sides, or the chest muscles on both sides, to collect the myoelectric data of the muscles. In practice, the sensor for collecting the myoelectric data may be a contact or non-contact myoelectric sensor, which will not be repeated here.

Please refer to FIG. 1 again. The sensing module 20 is coupled to the posture sensor 10 and is configured to output multiple limb torque data according to the posture data, and output multiple muscle group activation time data according to the myoelectric data. Specifically, the sensing module 20 outputs the limb torque data after performing spatial coordinate conversion according to the posture data sensed by the inertia sensor 110. In addition, the sensing module 20 outputs the muscle group activation time data after performing dynamic electromyography (EMG) processing according to the myoelectric data sensed by the myoelectric sensor 120a and the myoelectric sensor 120b.

The computing module 30 is coupled to the sensing module 20 and is configured to respectively convert the limb torque data and the muscle group activation time data into moment-skeleton coordinates and muscle strength eigenvalue-skeleton coordinates according to a skeleton coordinate system, perform fusion calculation on the moment-skeleton coordinates and the muscle strength eigenvalue-skeleton coordinates, calculate evaluation data for the training action according to a result of the fusion calculation, and judge that the training action corresponds to one of multiple known exercise actions according to the evaluation data. A detailed description will be given later. Practically speaking, the computing module 30 may be a central processing unit (CPU), a digital signal processor (DSP), multiple microprocessors, one or more microprocessors combined with a digital signal processor core, a controller, a microcontroller, an application specific integrated circuit (ASIC), a field programmable gate array (FPGA), any other type of integrated circuit, a state machine, a processor according to an advanced RISC machine (ARM), and the like, but not limited thereto.

The display module 40 is coupled to the computing module 30 and is configured to display the evaluation data and the known exercise action. In an embodiment, the display module 40 may display the evaluation data and the known exercise action in texts, icons, and graphs. In another embodiment, the display module 40 may further display the evaluation data and the known exercise action with audio and video together with texts, icons, and graphs. Practically speaking, the display module 40 may be an electronic device with a display function such as a monitor, a platform computer, or a personal computer, or a display device on a treadmill, but not limited thereto.

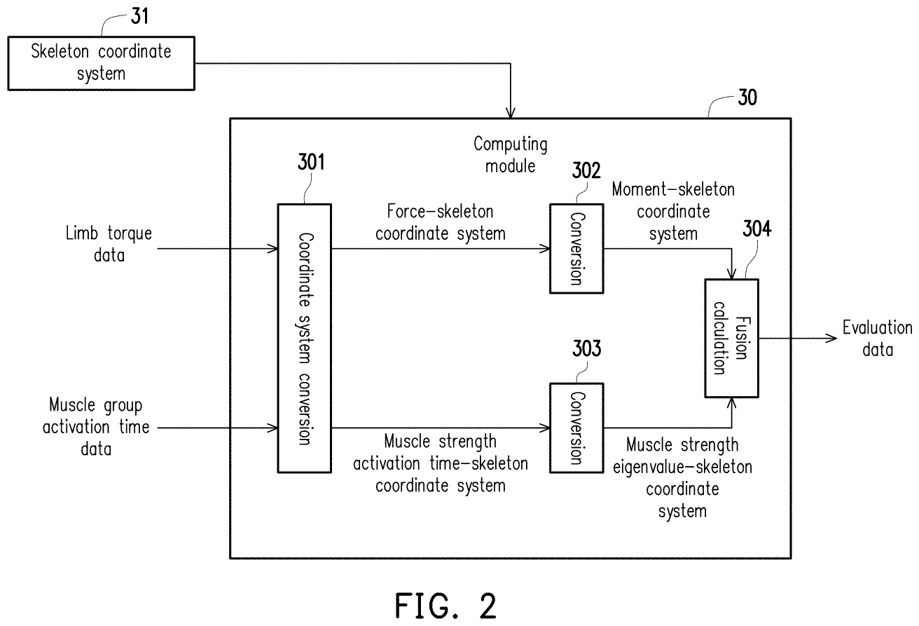

FIG. 2 is a schematic diagram of performing fusion calculation by the computing module 30 in the multiple sensor-fusing based interactive training system 1 according to an embodiment of the disclosure. Please refer to FIG. 1 and FIG. 2 at the same time. A skeleton coordinate system 31 is input to the computing module 30, and the limb torque data and the muscle group activation time data are synchronously and successively input to the computing module 30. The computing module 30 performs coordinate system conversion 301 on the limb torque data according to the skeleton coordinate system 31, and converts the limb torque data into a force-skeleton coordinate system. Afterwards, the computing module 30 performs conversion 302 to convert the force-skeleton coordinate system into a moment-skeleton coordinate system. The computing module 30 performs the coordinate system conversion 301 on the muscle group activation time data according to the skeleton coordinate system 31, and converts the muscle group activation time data into a muscle strength activation time-skeleton coordinate system. Afterwards, the computing module 30 performs conversion 303 to convert the muscle strength activation time-skeleton coordinate system into a muscle strength eigenvalue-skeleton coordinate system.

Next, the computing module 30 continues to perform fusion calculation 304 on the moment-skeleton coordinate system and the muscle strength eigenvalue-skeleton coordinate system, calculates evaluation data for the training actions of the user according to a result of the fusion calculation 304, and outputs the evaluation data to the display module 40.

Specifically, the evaluation data is a type of data for quantifying the exercise effectiveness of the user after the computing module 30 performs conversion and fusion calculation on the limb torque data and the muscle group activation time data. In an embodiment, the computing module 30 adopts K-mean clustering (KMC) to perform the fusion calculation 304 on the moment-skeleton coordinate system and the muscle strength eigenvalue-skeleton coordinate system.

As shown in FIG. 1, the computing module 30 judges that the training action of the user corresponds to one of multiple known exercise actions according to the evaluation data, and outputs the judged known exercise action to the display module 40. The display module 40 displays the evaluation data and the judged known exercise action for the user to observe whether the posture is accurate when performing the training action, whether the main muscle groups used match the training action, whether the sequence of straining of muscle groups for the training action is accurate, etc.

In an embodiment, the user may link with social data through the multiple sensor-fusing based interactive training system 1 described in the disclosure, and upload the evaluation data and the known exercise action to a social networking site or a training site. Alternatively, the user may interact with other users performing the same known exercise action regarding the evaluation data of each other on the social networking site or the training site. Alternatively, the user may conduct an online discussion with a remote exercise coach based on the evaluation data uploaded to the social networking site or the training site.

In an embodiment, if the training action being executed by the user is the same as the known exercise action judged by the computing module, it means that the posture data and the myoelectric data of the user conform to the display of the known exercise action, the user may confirm that the posture of the training action being executed is accurate, the main muscle groups used match the training action, the sequence of straining of muscle groups for the training action is accurate, etc.

In another embodiment, if the training action being executed by the user is different from the known exercise action judged by the computing module, it may mean that the posture of the user data and the myoelectric data do not completely conform to the display of the known exercise action, the user may further confirm or adjust the posture of the training action being executed, the main muscle groups used, the sequence of straining of muscle groups for the training action, etc. Alternatively, the user may further confirm whether the inertia sensor 110, the myoelectric sensor 120a, and the myoelectric sensor 120b are disposed at accurate positions.

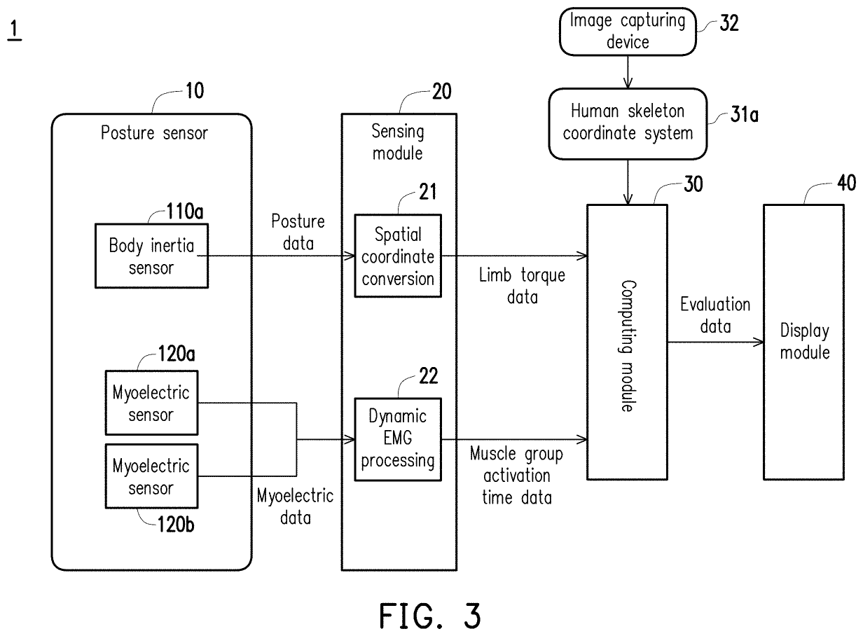

FIG. 3 is a block diagram of a multiple sensor-fusing based interactive training system 1 according to an embodiment of the disclosure. Please refer to FIG. 3. When the training action executed by the user does not require the use of training equipment, the posture sensor 10 includes a body inertia sensor 110a, a myoelectric sensor 120a, and a myoelectric sensor 120b. The body inertia sensor 110a is the same as the inertia sensor 110 disposed on the body or the limbs of the user, and the myoelectric sensor 120a and the myoelectric sensor 120b are the same as the myoelectric sensors attached or worn above the core muscle groups or related muscle groups of the user, which will not be repeated here.

In an embodiment, when the training action executed by the user does not require the use of training equipment, the body inertia sensor 110a outputs the posture data to the sensing module 20. The sensing module 20 outputs the limb torque data after performing spatial coordinate conversion 21 according to the posture data sensed by the body inertia sensor 110a. In addition, the sensing module 20 outputs multiple muscle group activation time data after performing dynamic EMG processing 22 according to the myoelectric data sensed by the myoelectric sensor 120a and the myoelectric sensor 120b.

Since the posture sensor 10 only includes the body inertia sensor 110a, the skeleton coordinate system input to the computing module 30 is a human skeleton coordinate system 31a. The limb torque data and the muscle group activation time data are synchronously and successively input to the computing module 30. The computing module 30 performs conversion and fusion calculation on the limb torque data and the muscle group activation time data according to the human skeleton coordinate system 31a, calculates the evaluation data, and outputs the evaluation data to the display module 40.

In an embodiment, when only the body inertia sensor 110a is disposed on the body of the user, the human skeleton coordinate system 31a corresponds to the skeleton of the user, including the bones, muscles, etc. of the human. The skeleton of the user may be obtained through an image capturing device 32.

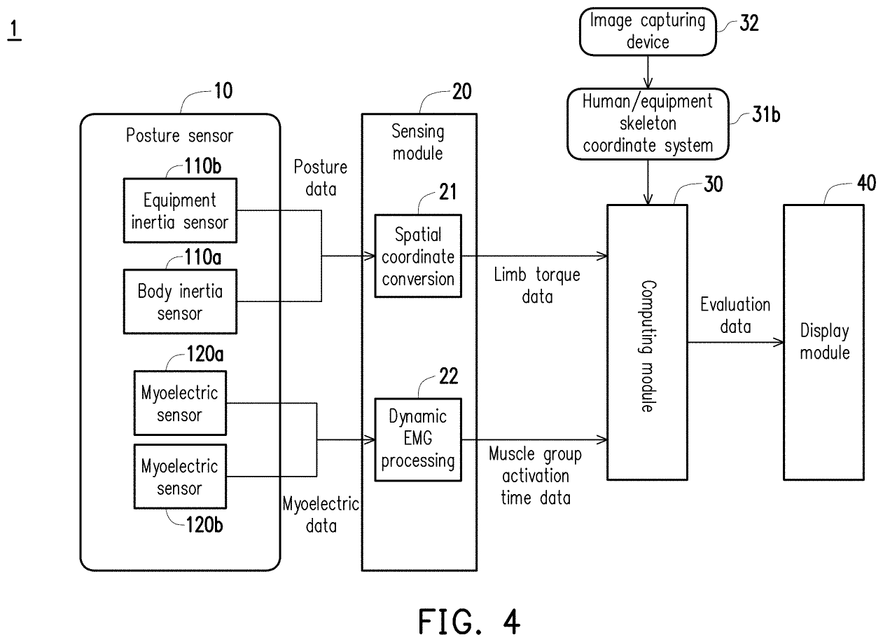

FIG. 4 is a block diagram of a multiple sensor-fusing based interactive training system 1 according to another embodiment of the disclosure. Please refer to FIG. 4. When the training action executed by the user requires the use of training equipment, the posture sensor 10 includes the body inertia sensor 110a, an equipment inertia sensor 110b, the myoelectric sensor 120a, and the myoelectric sensor 120b. The equipment inertia sensor 110b may be disposed on the training equipment such as a bat and a club used by the user when executing the training action depending on the training action of the user and is configured to sense the posture data related to the training action of the user. The body inertia sensor 110a is the same as the inertia sensor 110 disposed on the body or the limbs of the user, and the myoelectric sensor 120a and the myoelectric sensor 120b are the same as the myoelectric sensors attached or worn above the core muscle groups or related muscle groups of the user, which will not be repeated here.

In an embodiment, when the training action executed by the user requires the use of training equipment, the body inertia sensor 110a and the equipment inertia sensor 110b both output the posture data to the sensing module 20. The sensing module 20 outputs the limb torque data after performing the spatial coordinate conversion 21 according to the posture data sensed by the body inertia sensor 110a and the equipment inertia sensor 110b. In addition, the sensing module 20 outputs the muscle group activation time data after performing the dynamic EMG processing 22 according to the myoelectric data sensed by the myoelectric sensor 120a and the myoelectric sensor 120b.

Since the posture sensor 10 includes the body inertia sensor 110a and the equipment inertia sensor 110b at the same time, the skeleton coordinate system input to the computing module 30 is a human/equipment skeleton coordinate system 31b. The limb torque data and the muscle group activation time data are synchronously and successively input to the computing module 30. The computing module 30 performs conversion and fusion calculation on the limb torque data and the muscle group activation time data according to the human/equipment skeleton coordinate system 31b, calculates the evaluation data, and outputs the evaluation data to the display module 40.

In an embodiment, when the body inertia sensor 110a is disposed on the body of the user, and the equipment inertia sensor 110b is disposed on the training equipment at the same time, the human/equipment skeleton coordinate system 31b not only corresponds to the body skeleton of the user, including the bones, muscles, etc. of the human, but also corresponds to the equipment skeleton of the training equipment. The body skeleton of the user and the equipment skeleton of the training equipment may be both obtained through the image capturing device 32.

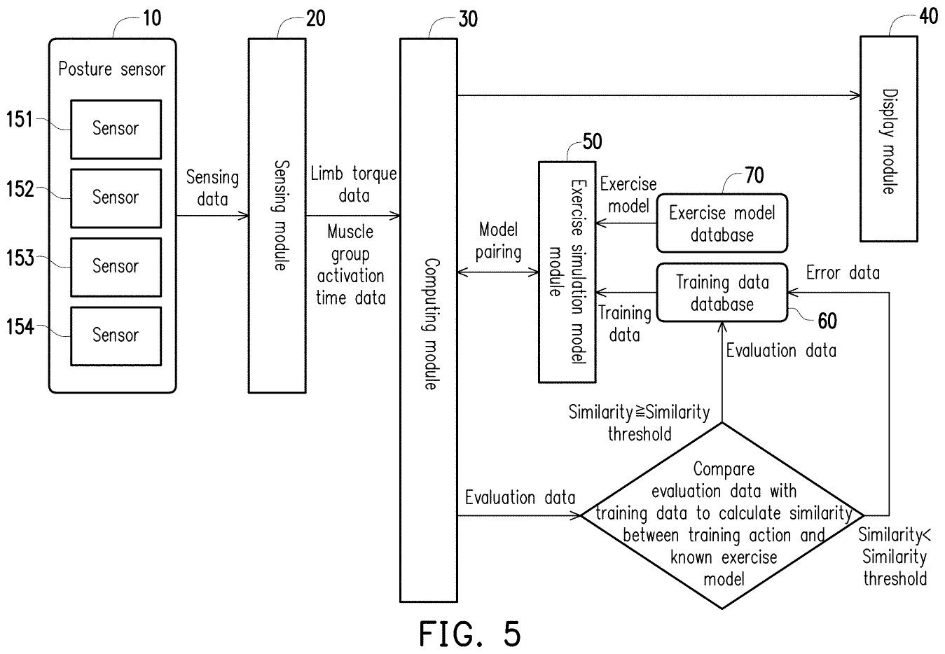

FIG. 5 is a block diagram of updating training data and error data by a multiple sensor-fusing based interactive training system 1 according to an embodiment of the disclosure. Please refer to FIG. 1 and FIG. 5 at the same time. The posture sensor 10 includes at least two sensors disposed on the user, wherein the sensor disposed on the user may be one of an inertia sensor and a myoelectric sensor or a combination thereof. As shown in FIG. 5, the posture sensor 10 includes a sensor 151, a sensor 152, a sensor 153, and a sensor 154, wherein the sensor 151, the sensor 152, the sensor 153, and the sensor 154 may respectively be an inertia sensor or a myoelectric sensor. In other words, the sensor 151, the sensor 152, the sensor 153, and the sensor 154 may all be inertia sensors or myoelectric sensors, or the sensor 151, the sensor 152, the sensor 153, and the sensor 154 may also partially be inertia sensors, while the remaining sensors are myoelectric sensors.

Please continue to refer to FIG. 5. The posture sensor 10 outputs multiple sensing data to the sensing module 20, wherein the sensing data corresponds to data output by the inertia sensor or the myoelectric sensor included in the posture sensor 10. The sensing module 20 outputs the limb torque data and/or the muscle group activation time data according to the sensing data. The computing module 30 performs conversion and fusion calculation on the limb torque data and the muscle group activation time data.

In an embodiment, the multiple sensor-fusing based interactive training system 1 further includes an exercise simulation model module 50, a training data database 60, and an exercise model database 70. The exercise simulation model module 50 is coupled to the computing module 30. The training data database 60 is coupled to the computing module 30 and the exercise simulation model module 50, and includes training data and error data corresponding to various known exercise models, wherein the error data is configured to judge whether the training action of the user is a wrong action or a dangerous action. The exercise model database 70 is coupled to the exercise simulation model module 50 and includes multiple known exercise models. The known exercise models are pre-established exercise models based on more than four inertia sensors or myoelectric sensors. In practice, the exercise simulation model module 50 may be a microprocessor or an embedded controller, and the training data database 60 and the exercise model database 70 may be storage media such as memories or hard disks, which are not limited in the disclosure.

The computing module 30 determines the exercise situation, such as running, aerobic exercise, and core muscle group training without equipment, of the user based on the number of posture sensors used by the user. After the computing module 30 determines the exercise situation of the user, exercise model pairing with the exercise simulation model module 50 is performed based on the exercise situation. The exercise simulation model module 50 selects one of the known exercise models from the exercise model database 70 based on the exercise situation. The purpose is to find an exercise model corresponding to the training action being executed by the user.

After the exercise simulation model module 50 selects a known exercise model corresponding to the training action being executed by the user from the exercise model database 70, the exercise simulation model module 50 reads training data corresponding to the selected known exercise model from the training data database 60 according to the selected known exercise model, and sends the training data back to the computing module 30. The computing module 30 compares the evaluation data with the training data corresponding to the selected known exercise model to calculate the similarity between the training action of the user and the selected known exercise model.

When the similarity is greater than or equal to a similarity threshold (for example, 0.5), the computing module 30 judges that the training action of the user conforms to the selected known exercise model, and stores the evaluation data to the training data database 60 to update the training data corresponding to the selected known exercise model to establish an artificial intelligence (AI) model. At the same time, the computing module 30 outputs the evaluation data and the known exercise action corresponding to the selected known exercise model to the display module 40. The display module 40 further displays the evaluation data and the known exercise action.

Conversely, when the similarity is less than the similarity threshold (for example, 0.5), the computing module 30 judges that the training action of the user does not conform to all the known exercise models included in the exercise model database 70. At this time, the computing module 30 stores the evaluation data to the training data database 60 to update the error data. At the same time, the computing module 30 outputs the evaluation data and a wrong exercise action message to the display module 40. The display module 40 further displays the evaluation data and the wrong exercise action message. The wrong exercise action message is configured to prompt the user to further confirm or adjust the posture of the training action being executed, the main muscle groups used, the sequence of straining of muscle groups for the training action, etc. Alternatively, the user may further confirm whether the posture sensor 10 is disposed at an accurate position.

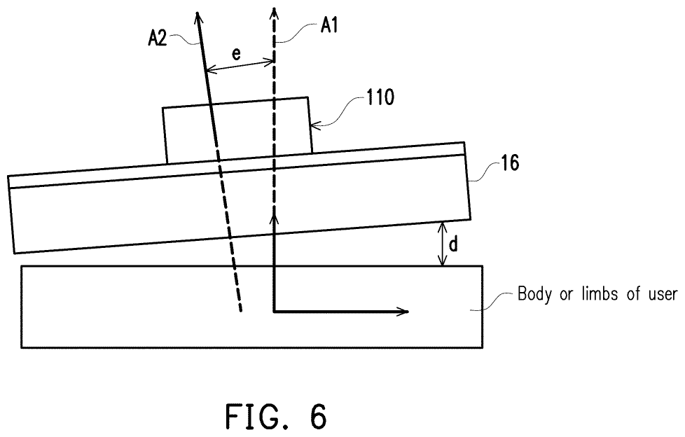

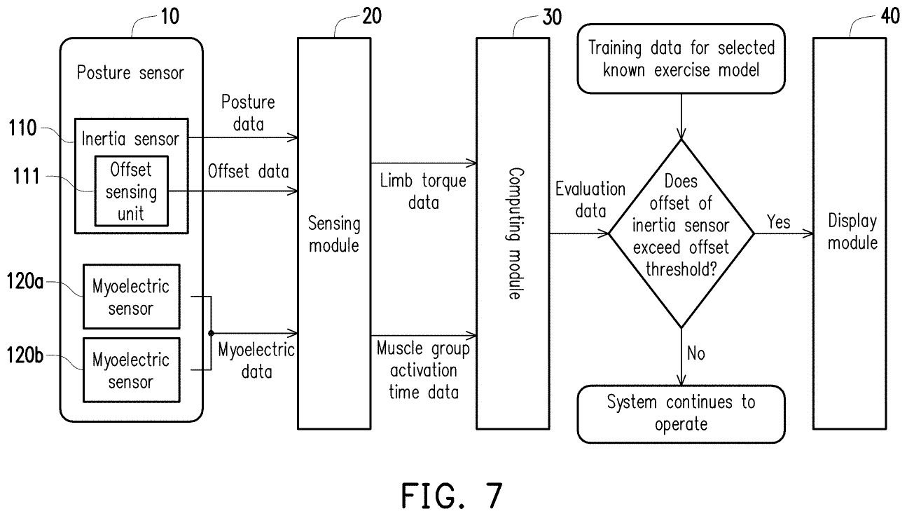

FIG. 6 is a schematic diagram of judging an offset using an inertia sensor 110 (for example, a gravity sensor) in a multiple sensor-fusing based interactive training system according to an embodiment of the disclosure. FIG. 7 is a block diagram of judging an offset using an inertia sensor 110 in a multiple sensor-fusing based interactive training system according to an embodiment of the disclosure. Please refer to FIG. 6 first. When the inertia sensor 110 is fixed on a sensing carrier 16, and the sensing carrier 16 is disposed on the body, the limbs, or the clothing of the user by means of strapping or wearing, during the exercise process, the inertia sensor 110 may be offset due to the shaking of the body or the swinging of the limbs of the user. Once there is a relative offset d between the inertia sensor 110 and the body or the limbs of the user, the accuracy of the posture data will be affected.

Please also refer to FIG. 6 and FIG. 7. The inertia sensor 110 has an offset sensing unit 111. When the sensing carrier 16 fixed with the inertia sensor 110 is attached to the body or the limbs of the user, the sensing data measured by the inertia sensor 110 is an acceleration A1, and the offset sensing unit 111 sets the acceleration A1 as a standard reference value. When there is the relative offset d between one side of the sensing carrier 16 fixed with the inertia sensor 110 and the body or the limbs of the user, the sensing data measured by the inertia sensor 110 is an acceleration A2. Once there is an error e between the acceleration A2 and the acceleration A1, the offset sensing unit 111 senses offset data corresponding to the acceleration A2 and outputs the offset data to the sensing module 20.

The sensing module 20 outputs the limb torque data to the computing module 30 according to the posture data and the offset data. The computing module 30 compares the evaluation data with the training data corresponding to the selected known exercise model, and judges whether the relative offset d between the sensing carrier 16 fixed with the inertia sensor 110 and the body of the user exceeds an offset threshold. When the relative offset d is not greater than the offset threshold, it means that the degree of offset of the sensing carrier 16 does not affect the accuracy of the posture data, and the multiple sensor-fusing based interactive training system 1 continues to operate. On the contrary, when the relative offset d is greater than the offset threshold, it means that the degree of offset of the sensing carrier 16 already affects the accuracy of the posture data, the computing module 30 outputs an abnormal signal to the display module 40, and the display module 40 displays a sensor setting abnormal message.

When the user is using the exercise equipment, a mechanical sensor may also be disposed on the exercise equipment to sense a straining state of the user when executing the training action, and detect whether the straining states of the left and right sides of the body of the user are balanced. Once the straining states of the left and right sides of the body of the user are unbalanced, the multiple sensor-fusing based interactive training system of the disclosure can further issue a warning to prompt the user to pay attention to the unbalanced straining states of the left and right sides of the body.

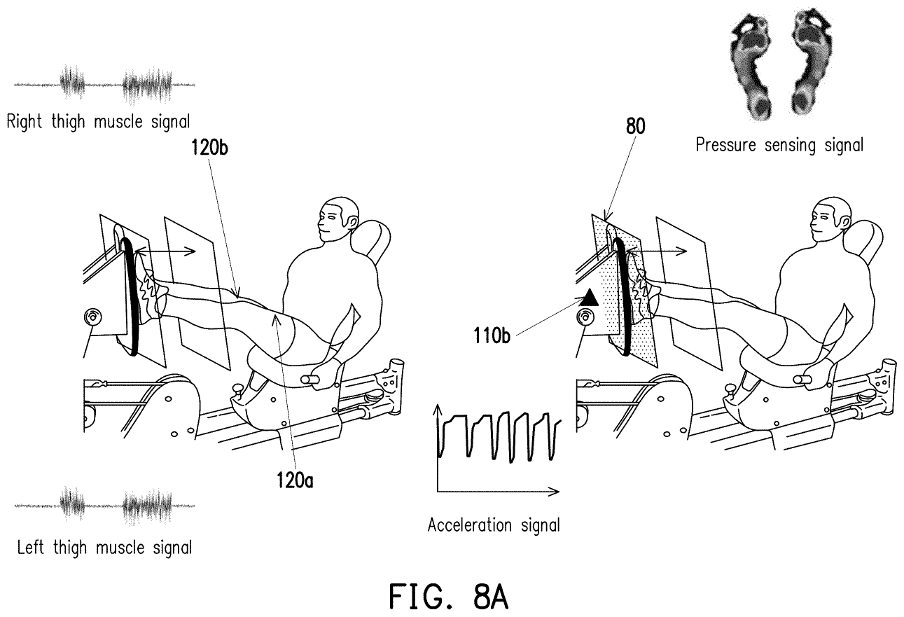

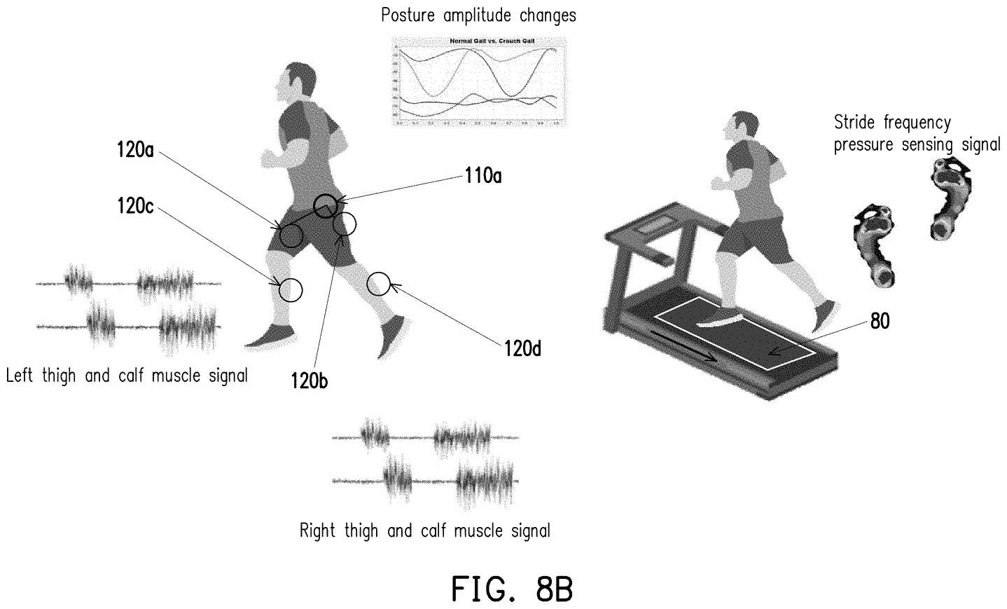

FIG. 8A is a schematic diagram of calculating left-right balance using a posture sensor and a mechanical sensor 80 in a multiple sensor-fusing based interactive training system according to an embodiment of the disclosure. As shown in FIG. 8A, when the user uses the training equipment in FIG. 8A, the user needs to push the platform on the fitness equipment with both feet at the same time. The multiple sensor-fusing based interactive training system further includes the mechanical sensor 80. The mechanical sensor 80 is disposed on the training equipment and is coupled to the sensing module, and is configured to sense multiple mechanical data (for example, pressure sensing signals) corresponding to the training action of the users. When the user pushes the platform on the fitness equipment with both feet at the same time, the mechanical sensor 80 senses the mechanical data (for example, the pressure sensing signals) corresponding to the training action of the user, wherein the mechanical data may be a total pressure sensing signal corresponding to both feet of the user pushing the platform at the same time or respectively be a pressure sensing signal corresponding to the left foot of the user and a pressure sensing signal corresponding to the right foot of the user depending on the configuration manner or the number of the mechanical sensor 80.

Please continue to refer to FIG. 8A. When the user is using the exercise equipment, the myoelectric sensor 120a and the myoelectric sensor 120b are respectively disposed on the left half and the right half of the body of the user, and the equipment inertia sensor 110b is disposed on the exercise equipment. The myoelectric sensor 120a and the myoelectric sensor 120b are configured to sense multiple left half myoelectric data (for example, left thigh muscle signals) and multiple right half myoelectric data (for example, right thigh muscle signals) corresponding to the training action of the user, and the equipment inertia sensor 110b is configured to sense multiple posture data (for example, acceleration signals) related to the training action of the user.

FIG. 8B is another schematic diagram of calculating left-right balance using a posture sensor and a mechanical sensor 80 in a multiple sensor-fusing based interactive training system according to an embodiment of the disclosure. As shown in FIG. 8B, when the user is running, since the running action is to step on the ground with both feet in an alternating manner, that is, only one foot touches the ground at a time, the balance of straining of both legs affects the effectiveness of running, and even affects the safety of running. Therefore, it is necessary to detect the left-right balance respectively for both legs of the user.

The myoelectric sensor 120a and a myoelectric sensor 120c are respectively disposed on the left thigh and the left calf of the body of the user, the myoelectric sensor 120b and a myoelectric sensor 120d are respectively disposed on the right thigh and the right calf of the body of the user, and the body inertia sensor 110a is disposed on the rear side of the waist of the user. The myoelectric sensor 120a and the myoelectric sensor 120c are configured to sense multiple left half myoelectric data (for example, left thigh and calf muscle signals) corresponding to the training action of the user, the myoelectric sensor 120b and the myoelectric sensor 120d are configured to sense multiple right half myoelectric data (for example, right thigh and calf muscle signals) corresponding to the training action of the user, and the body inertia sensor 110a is configured to sense multiple posture data (for example, stride length, stride frequency, vertical amplitude, body inclination angle, feet contact time, feet movement trajectory, and other posture amplitude changes) related to the training action of the user.

As shown in FIG. 8B, when the user uses the training equipment (that is, the treadmill) in FIG. 8B, both feet of the user step on the belt of the treadmill in an alternating manner. The mechanical sensor 80 is disposed on the belt of the treadmill and is coupled to the sensing module, and is configured to sense multiple mechanical data (for example, pressure sensing signals) corresponding to the training action of the user. Since the user does not put both feet on the belt of the treadmill at the same time when running, the mechanical sensor 80 may sense multiple mechanical data (for example, stride frequency pressure sensing signals) corresponding to the training action of the user). In particular, the mechanical data respectively correspond to the stride frequency pressure sensing signal of the left foot of the user and the stride frequency pressure sensing signal of the right foot of the user.

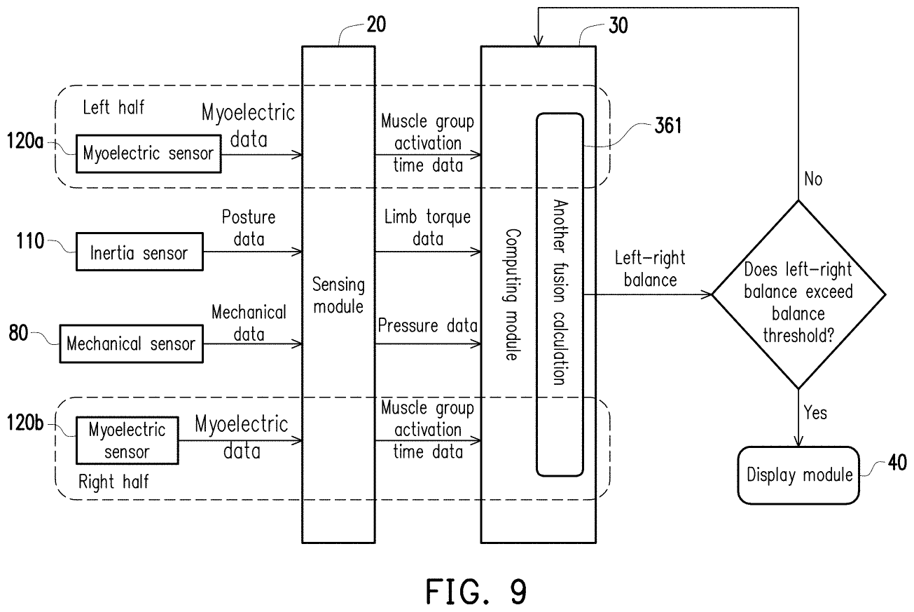

FIG. 9 is a block diagram of calculating left-right balance using a posture sensor and a mechanical sensor 80 in a multiple sensor-fusing based interactive training system according to an embodiment of the disclosure. The sensing module 20 outputs multiple pressure data according to multiple mechanical data, and outputs multiple limb torque data according to multiple posture data. At the same time, the sensing module 20 respectively outputs multiple left half muscle group activation time data and multiple right half muscle group activation time data according to the left half myoelectric data and the right half myoelectric data. The computing module 30 calculates a left half straining value according to the pressure data, the limb torque data, and the left half muscle group activation time data, and calculates a right half straining value according to the pressure data, the limb torque data, and the right half muscle group activation time data at the same time. Then, the computing module 30 performs another fusion calculation 361 on left half straining data and right half straining data, and calculates the left-right balance corresponding to the training action of the user according to a result of the another fusion calculation 361.

When the left-right balance is less than or equal to a balance threshold, the computing module 30 judges that the straining of the left half and the right half of the body of the user is balanced, and continues to calculate the left-right balance corresponding to the training action of the user according to the result of another fusion calculation. Conversely, when the left-right balance is greater than the balance threshold, the computing module 30 judges that the straining of the left half and the right half of the body of the user is unbalanced, and the display module 40 displays the evaluation data and an unbalance message to prompt the user to pay attention to the unbalanced state of the straining of the left and right sides of the body.

As shown in FIG. 1, in an embodiment, the multiple sensor-fusing based interactive training system 1 further includes a physiological information sensor 90, which is coupled to the sensing module 20 and is configured to sense multiple physiological data of the user, such as information data such as body temperature, heart rate, respiration, skin moisture content, and sweat when the user is performing the training action, and send the physiological data to the computing module 30. The computing module 30 may monitor the physiological condition of the user when performing the training action based on the physiological data, and judge whether to issue a warning signal to prompt the user to stop the training action. In practice, the physiological information sensor 90 may be a sensor that acquires the physiological values by contact or non-contact, which is not limited in the disclosure.

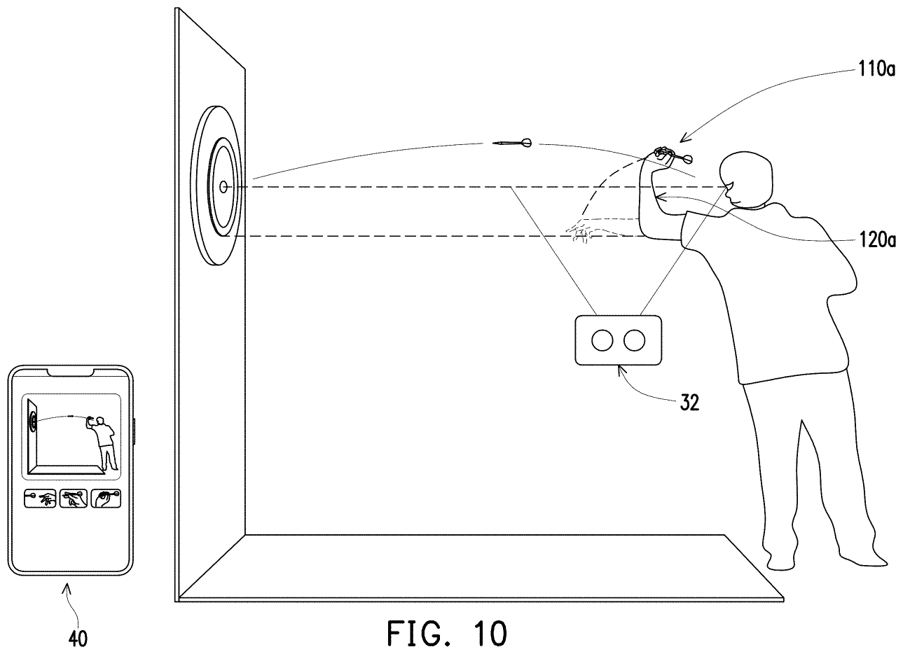

FIG. 10 is a schematic diagram of performing action recognition using a body inertia sensor 110a and a myoelectric sensor 120a in a multiple sensor-fusing based interactive training system according to an embodiment of the disclosure. Please refer to FIG. 10. In an embodiment, the user may perform the action recognition using the multiple sensor-fusing based interactive training system. The body inertia sensor 110a is disposed on a glove, and the myoelectric sensor 120a is disposed on a wrist strap.

When the user intends to execute a dart-throwing action, the glove equipped with the body inertia sensor 110a may be worn on the dart-throwing hand of the user, and the wrist strap equipped with the myoelectric sensor 120a may be fixed on the dart-throwing wrist of the user. The body inertia sensor 110a is configured to sense multiple posture data related to the action of the hand when the user throws darts. The myoelectric sensor 120a is configured to sense multiple myoelectric data related to the user throwing darts.

In addition, the image capturing device 32 is configured to obtain a postural body image of the user when throwing darts. When the user throws the dart, the dart-throwing hand of the user first lifts, stretches back, and then throws the dart forward, so the postural body image of the user throwing the dart includes the movement trajectory of the hand of the user. In addition, when the user throws the dart, the body of the user may also use the force of body rotation to throw the dart, so the postural body image of the user when throwing the dart also includes the rotational trajectory of the body of the user.

Next, please refer to FIG. 3 and FIG. 10 at the same time. The body inertia sensor 110a outputs posture data to the sensing module 20. The sensing module 20 outputs the limb torque data after performing the spatial coordinate conversion 21 according to the posture data sensed by the body inertia sensor 110a. In addition, the sensing module 20 outputs the muscle group activation time data after performing the dynamic EMG processing 22 according to the myoelectric data sensed by the myoelectric sensor 120a.

The posture sensor 10 includes both the body inertia sensor 110a and the myoelectric sensor 120a, so the skeleton coordinate system input to the computing module 30 is the human skeleton coordinate system 31a. The limb torque data and the muscle group activation time data are synchronously and successively input to the computing module 30. The computing module 30 performs conversion and fusion calculation on the limb torque data and the muscle group activation time data according to the human skeleton coordinate system 31a, calculates the evaluation data, and outputs the evaluation data to the display module 40.

In addition, in an embodiment, the computing module 30 may output the postural body image of the user when throwing darts to the display module 40 (for example, a mobile device). The user may watch the postural body image through the display module 40, or even watch the movement trajectory of the hand and the rotational trajectory of the body through slowing down the postural body image. The computing module 30 may also be combined with an AI action analysis module to execute action analysis on the postural body image of the user when throwing darts, and quantify the exercise effectiveness in combination with the evaluation data to provide the user with adjustment suggestions for the hand action and the body rotation.

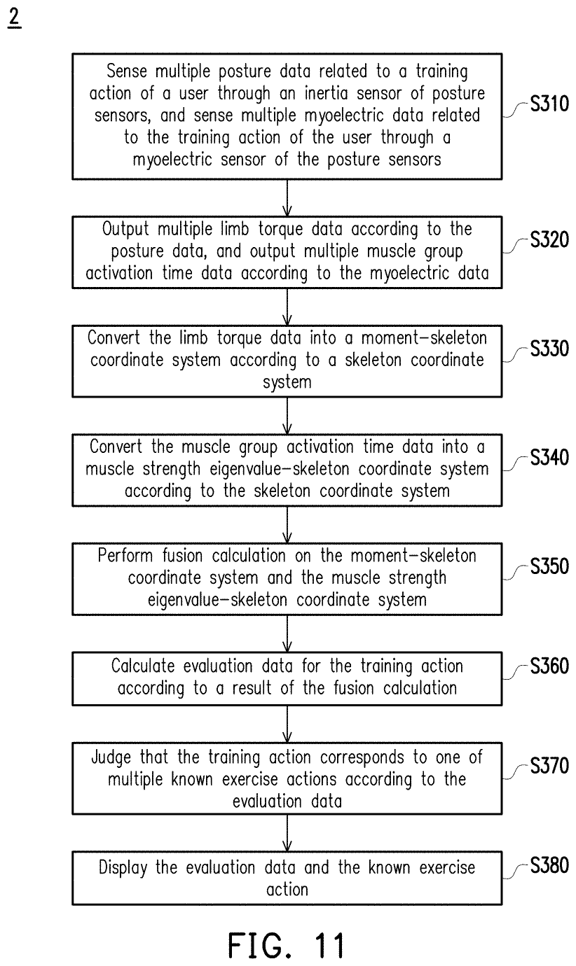

FIG. 11 is a flowchart of a multiple sensor-fusing based interactive training method 2 according to an embodiment of the disclosure. As shown in FIG. 11, the multiple sensor-fusing based interactive training method 2 includes Steps S310 to S380.

In Step S310, multiple posture data related to a training action of a user is sensed through at least one inertia sensor of multiple posture sensors, and multiple myoelectric data related to the training action of the user is sensed through at least one myoelectric sensor of the posture sensors. In Step S320, multiple limb torque data are output according to the posture data, and multiple muscle group activation time data are output according to the myoelectric data.

In Step S330, the limb torque data are converted into a moment-skeleton coordinate system according to a skeleton coordinate system. In Step S340, the muscle group activation time data are converted into a muscle strength eigenvalue-skeleton coordinate system according to the skeleton coordinate system. The disclosure does not limit the execution sequence of Step S330 and Step S340, and Step S330 and Step S340 may also be performed at the same time. In Step S350, fusion calculation is performed on the moment-skeleton coordinate system and the muscle strength eigenvalue-skeleton coordinate system. The fusion calculation is performed by adopting K-mean clustering (KMC) on the moment-skeleton coordinate system and the muscle strength eigenvalue-skeleton coordinate system. In Step S360, evaluation data is calculated for the training action according to a result of the fusion calculation.

In Step S370, the training action corresponds to one of multiple known exercise actions is judged according to the evaluation data. In Step S380, the evaluation data and the known exercise action are displayed.

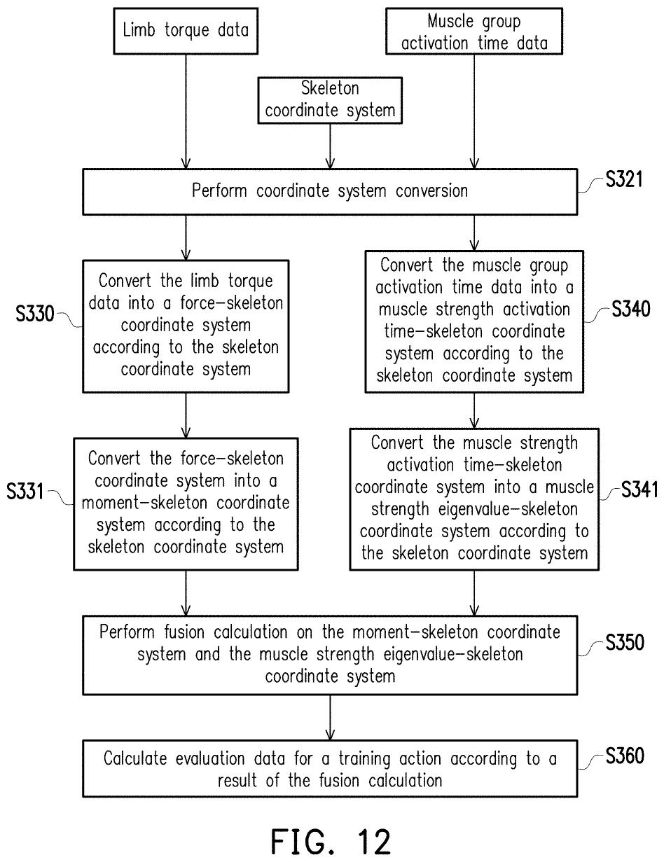

FIG. 12 is a flowchart of calculating evaluation data in a multiple sensor-fusing based interactive training method according to an embodiment of the disclosure. As shown in FIG. 12. In Step S321, coordinate system conversion is performed on multiple limb torque data and multiple muscle group activation time data according to a skeleton coordinate system. In Step S330, the limb torque data are converted into a force-skeleton coordinate system according to the skeleton coordinate system. Then, in Step S331, the force-skeleton coordinate system is converted into a moment-skeleton coordinate system according to the skeleton coordinate system. In addition, in Step S340, the muscle group activation time data are converted into a muscle strength activation time-skeleton coordinate system according to the skeleton coordinate system. Then, in Step S341, the muscle strength activation time-skeleton coordinate system is converted into a muscle strength eigenvalue-skeleton coordinate system according to the skeleton coordinate system. It should be particularly noted that Step S330 to Step S331 and Step S340 to Step S341 are two separate processes, which may be performed at the same time or separately.

Once the moment-skeleton coordinate system and the muscle strength eigenvalue-skeleton coordinate system are converted according to the skeleton coordinate system, in Step S350, fusion calculation is performed on the moment-skeleton coordinate system and the muscle strength eigenvalue-skeleton coordinate system. Next, in Step S360, evaluation data is calculated for the training action according to a result of the fusion calculation.

In an embodiment, when the inertia sensor is disposed on the body of the user, the skeleton coordinate system corresponds to the skeleton of the user, and the skeleton of the user may be obtained through the image capturing device. In an embodiment, when the inertia sensor is disposed on the training equipment, the skeleton coordinate system further corresponds to the skeleton of the training equipment, and the skeleton of the training equipment may be obtained through the image capturing device.

In an embodiment, the multiple sensor-fusing based interactive training method further includes determining an exercise situation of the user based on the number of posture sensors used by the user, and selecting one of multiple known exercise models based on the exercise situation.

In an embodiment, the multiple sensor-fusing based interactive training method further includes comparing the evaluation data with the training data corresponding to the selected known exercise model to calculate the similarity between the training action of the user and the selected known exercise model. When the similarity is greater than or equal to the similarity threshold, the training action of the user conforms to the selected known exercise model is judged. The training data corresponding to the selected known exercise model is updated with the evaluation data, and the evaluation data and the known exercise action are displayed. Conversely, when the similarity is less than the similarity threshold, the training action of the user does not conform to each of all the known exercise models is judged. The evaluation data is updated to the error data, and the evaluation data and the wrong exercise action message are displayed.

In an embodiment, the inertia sensor has the offset sensing unit, and the inertia sensor is disposed on the body of the user. The multiple sensor-fusing based interactive training method further includes when there is the relative offset between the inertia sensor and the body of the user, the offset data are sensed, and multiple limb torque data are output according to the posture data and the offset data. The evaluation data is compared with the training data corresponding to the selected known exercise model, and whether the relative offset between the inertia sensor and the body of the user exceeds the offset threshold is judged. When the relative offset is greater than the offset threshold, the sensor setting abnormal message is displayed.

In an embodiment, the multiple sensor-fusing based interactive training method further includes sensing the mechanical data corresponding to the training action of the user through the mechanical sensors respectively disposed on the training equipment, sensing the posture data corresponding to the training action of the user through at least one inertia sensor disposed on the training equipment, and sensing the left half myoelectric data and the right half myoelectric data through at least two myoelectric sensors respectively disposed on the left half and the right half of the body of the user. The pressure data are output according to the mechanical data, the limb torque data are output according to the posture data, and the left half muscle group activation time data and the right half muscle group activation time data are respectively output according to the left half myoelectric data and the right half myoelectric data. The left half straining value is calculated according to the pressure data, the limb torque data, and the left half muscle group activation time data, and the right half straining value is calculated according to the pressure data, the limb torque data, and the right half muscle group activation time data. Another fusion calculation is performed on the left half straining data and the right half straining data, and the left-right balance corresponding to the training action of the user is calculated according to the result of the another fusion calculation.

In an embodiment, when the left-right balance is less than or equal to the balance threshold, the straining of the left half and the right half of the body of the user is balanced is judged, and the left-right balance corresponding to the training action of the user is continued to be calculated according to the result of the another fusion calculation. Conversely, when the left-right balance is greater than the balance threshold, the straining of the left half and the right half of the body of the user is unbalanced is judged, and the evaluation data and the unbalance message are displayed.

In an embodiment, the multiple sensor-fusing based interactive training method further includes sensing the physiological data of the user, such as the information data such as body temperature, heart rate, respiration, skin moisture content, and sweat when the user is performing the training action. The physiological condition of the user when performing the training action is monitored based on the physiological data, and whether to issue the warning signal to prompt the user to stop the training action is judged.

In summary, the multiple sensor-fusing based interactive training system and the multiple sensor-fusing based interactive training method provided by the disclosure can provide a gym user without the guidance of a coach to know whether the posture of using the fitness equipment is accurate, whether the muscle groups used match the training action, or whether the sequence of straining of muscle groups for the training action is accurate, which can avoid exercise injuries, and can also quantify the exercise effectiveness as an indicator for the coach and the athlete to discuss improvement manners.

It will be apparent to those skilled in the art that various modifications and variations may be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A multiple sensor-fusing based interactive training system, comprising:
a plurality of posture sensors, comprising:
at least one inertia sensor, configured to sense a plurality of posture data related to a training action of a user; and
at least one myoelectric sensor, configured to sense a plurality of myoelectric data related to the training action of the user;
a sensing module, coupled to the posture sensors and configured to output a plurality of limb torque data according to the posture data, and output a plurality of muscle group activation time data according to the myoelectric data;
a computing module, coupled to the sensing module and configured to execute:
converting the limb torque data into a moment-skeleton coordinate system according to a skeleton coordinate system;
converting the muscle group activation time data into a muscle strength eigenvalue-skeleton coordinate system according to the skeleton coordinate system;
performing fusion calculation on the moment-skeleton coordinate system and the muscle strength eigenvalue-skeleton coordinate system;
calculating evaluation data for the training action according to a result of the fusion calculation; and
judging that the training action corresponds to one of a plurality of known exercise actions according to the evaluation data; and
a display module, coupled to the computing module and configured to display the evaluation data and the known exercise action.

2. The multiple sensor-fusing based interactive training system according to claim 1, wherein the computing module is further configured to execute:
converting the limb torque data into a force-skeleton coordinate system according to the skeleton coordinate system, and then converting the force-skeleton coordinate system into the moment-skeleton coordinate system; and
converting the muscle group activation time data into a muscle strength activation time-skeleton coordinate system according to the skeleton coordinate system, and then converting the muscle strength activation time-skeleton coordinate system into the muscle strength eigenvalue-skeleton coordinate system.

3. The multiple sensor-fusing based interactive training system according to claim 1,
wherein when the at least one inertia sensor is disposed on a body of the user, the skeleton coordinate system corresponds to a body skeleton of the user, and the body skeleton of the user is obtained through an image capturing device.

4. The multiple sensor-fusing based interactive training system according to claim 3, wherein when the at least one inertia sensor is disposed on a training equipment, the skeleton coordinate system further corresponds to an equipment skeleton of the training equipment, and the equipment skeleton of the training equipment is obtained through the image capturing device.

5. The multiple sensor-fusing based interactive training system according to claim 1, further comprising:
an exercise model database, comprising a plurality of known exercise models; and
an exercise simulation model module, coupled to the exercise model database and the computing module,
wherein the computing module determines an exercise situation of the user based on a number of the posture sensors used by the user,
wherein the computing module performs pairing with the exercise simulation model module based on the exercise situation, and the exercise simulation model module selects one of the known exercise models from the exercise model database based on the exercise situation.

6. The multiple sensor-fusing based interactive training system according to claim 5, further comprising:
a training data database, coupled to the computing module, the training data database comprising training data and error data corresponding to each of the known exercise models,
wherein the computing module compares the evaluation data with the training data corresponding to the selected known exercise model to calculate a similarity between the training action of the user and the selected known exercise model.

7. The multiple sensor-fusing based interactive training system according to claim 6, wherein:
when the similarity is greater than or equal to a similarity threshold, the computing module judges that the training action of the user conforms to the selected known exercise model, and stores the evaluation data to the training data database to update the training data corresponding to the selected known exercise model, and the display module displays the evaluation data and the known exercise action;
when the similarity is less than a similarity threshold, the computing module judges that the training action of the user does not conform to the known exercise models, and stores the evaluation data to the training data database to update the error data, and the display module displays the evaluation data and a wrong exercise action message.

8. The multiple sensor-fusing based interactive training system according to claim 5, wherein the at least one inertia sensor has an offset sensing unit, the at least one inertia sensor is disposed on a body of the user and is configured to sense a plurality of offset data when there is a relative offset between the at least one inertia sensor and the body of the user, and the sensing module outputs the limb torque data according to the posture data and the offset data,
wherein the computing module compares the evaluation data with the training data corresponding to the selected known exercise model, and judges whether the relative offset between the at least one inertia sensor and the body of the user exceeds an offset threshold,
wherein when the relative offset is greater than the offset threshold, the display module displays a sensor setting abnormal message.

9. The multiple sensor-fusing based interactive training system according to claim 1, further comprising:
a mechanical sensor, coupled to the sensing module, disposed on a training equipment, and configured to sense a plurality of mechanical data corresponding to the training action of the user,
wherein the at least one inertia sensor is disposed on the training equipment and is configured to sense the posture data corresponding to the training action of the user,
wherein the at least one myoelectric sensor is respectively disposed on a left half and a right half of a body of the user and configured to sense a plurality of left half myoelectric data and a plurality of right half myoelectric data corresponding to the training action of the user,
wherein the sensing module outputs a plurality of pressure data according to the mechanical data, and outputs the limb torque data according to the posture data,
wherein the sensing module respectively outputs a plurality of left half muscle group activation time data and a plurality of right half muscle group activation time data according to the left half myoelectric data and the right half myoelectric data,
wherein the computing module is further configured to execute:
calculating a left half straining value according to the pressure data, the limb torque data, and the left half muscle group activation time data;
calculating a right half straining value according to the pressure data, the limb torque data, and the right half muscle group activation time data;
performing another fusion calculation on left half straining data and right half straining data; and
calculating left-right balance corresponding to the training action of the user according to a result of the another fusion calculation.

10. The multiple sensor-fusing based interactive training system according to claim 9, wherein:
when the left-right balance is less than or equal to a balance threshold, the computing module judges that straining of the left half and the right half of the body of the user is balanced, and continues to calculate the left-right balance corresponding to the training action of the user according to a result of the another fusion calculation; and
when the left-right balance is greater than the balance threshold, the computing module judges that straining of the left half and the right half of the body of the user is unbalanced, and the display module displays the evaluation data and an unbalance message.

11. A multiple sensor-fusing based interactive training method, comprising:
sensing a plurality of posture data related to a training action of a user through at least one inertia sensor of a plurality of posture sensors, and sensing a plurality of myoelectric data related to the training action of the user through at least one myoelectric sensor of the posture sensors;
outputting a plurality of limb torque data according to the posture data, and outputting a plurality of muscle group activation time data according to the myoelectric data;
converting the limb torque data into a moment-skeleton coordinate system according to a skeleton coordinate system;
converting the muscle group activation time data into a muscle strength eigenvalue-skeleton coordinate system according to the skeleton coordinate system;
performing fusion calculation on the moment-skeleton coordinate system and the muscle strength eigenvalue-skeleton coordinate system;
calculating evaluation data for the training action according to a result of the fusion calculation;
judging that the training action corresponds to one of a plurality of known exercise actions according to the evaluation data; and
displaying the evaluation data and the known exercise action.

12. The multiple sensor-fusing based interactive training method according to claim 11, further comprising:
converting the limb torque data into a force-skeleton coordinate system according to the skeleton coordinate system, and then converting the force-skeleton coordinate system into the moment-skeleton coordinate system; and
converting the muscle group activation time data into a muscle strength activation time-skeleton coordinate system according to the skeleton coordinate system, and then converting the muscle strength activation time-skeleton coordinate system into the muscle strength eigenvalue-skeleton coordinate system.

13. The multiple sensor-fusing based interactive training method according to claim 11, wherein when the at least one inertia sensor is disposed on a body of the user, the skeleton coordinate system corresponds to a body skeleton of the user, and the body skeleton of the user is obtained through an image capturing device.

14. The multiple sensor-fusing based interactive training method according to claim 13, wherein when the at least one inertia sensor is disposed on a training equipment, the skeleton coordinate system further corresponds to an equipment skeleton of the training equipment, and the equipment skeleton of the training equipment is obtained through the image capturing device.

15. The multiple sensor-fusing based interactive training method according to claim 11, further comprising:
determining an exercise situation of the user based on a number of the posture sensors used by the user;
selecting one of a plurality of known exercise models based on the exercise situation.

16. The multiple sensor-fusing based interactive training method according to claim 15, further comprising:
comparing the evaluation data with training data corresponding to the selected known exercise model to calculate a similarity between the training action of the user and the selected known exercise model.

17. The multiple sensor-fusing based interactive training method according to claim 16, wherein:
when the similarity is greater than or equal to a similarity threshold, the training action of the user conforms to the selected known exercise model is judged, the training data corresponding to the selected known exercise model is updated with the evaluation data, and the evaluation data and the known exercise action are displayed;
when the similarity is less than a similarity threshold, the training action of the user does not conform to the known exercise models is judged, the evaluation data is updated as error data, and the evaluation data and a wrong exercise action message are displayed.

18. The multiple sensor-fusing based interactive training method according to claim 15, wherein the at least one inertia sensor has an offset sensing unit, the at least one inertia sensor is disposed on a body of the user, and the multiple sensor-fusing based interactive training method further comprises:
when there is a relative offset between the at least one inertia sensor and the body of the user, sensing a plurality of offset data, and outputting the limb torque data according to the posture data and the offset data; and
comparing the evaluation data with the training data corresponding to the selected known exercise model, and judging whether the relative offset between the at least one inertia sensor and the body of the user exceeds an offset threshold,
wherein when the relative offset is greater than the offset threshold, a sensor setting abnormal message is displayed.

19. The multiple sensor-fusing based interactive training method according to claim 11, further comprising:
sensing a plurality of mechanical data corresponding to the training action of the user through a mechanical sensor disposed on a training equipment;
sensing the posture data corresponding to the training action of the user through the at least one inertia sensor disposed on the training equipment;
sensing a plurality of left half myoelectric data and a plurality of right half myoelectric data corresponding to the training action of the user through the at least one myoelectric sensor respectively disposed on a left half and a right half of a body of the user;
outputting a plurality of pressure data according to the mechanical data, and outputting the limb torque data according to the posture data;
respectively outputting a plurality of left half muscle group activation time data and a plurality of right half muscle group activation time data according to the left half myoelectric data and the right half myoelectric data;
calculating a left half straining value according to the pressure data, the limb torque data, and the left half muscle group activation time data;
calculating a right half straining value according to the pressure data, the limb torque data, and the right half muscle group activation time data;
performing another fusion calculation on left half straining data and right half straining data; and
calculating left-right balance corresponding to the training action of the user according to a result of the another fusion calculation.

20. The multiple sensor-fusing based interactive training method according to claim 19, wherein:
when the left-right balance is less than or equal to a balance threshold, straining of the left half and the right half of the body of the user is balanced is judged, and the left-right balance corresponding to the training action of the user is continued to be calculated according to a result of the another fusion calculation; and
when the left-right balance is greater than the balance threshold, straining of the left half and the right half of the body of the user is unbalanced is judged, and the evaluation data and an unbalance message are displayed.
